(19) United States
(12) Patent Application Publication
(10) Pub. No.: US 2009/0312620 A1
(43) Pub. Date: Dec. 17, 2009

(54) Diabetes monitor

(76) Inventor: Hou-Mei Henry Chang, Houston, TX (US)

Correspondence Address: Hou-Mei Henry Chang, 8934 Eastcove Circle, Houston, TX, 77064, US

(21) Appl. No.: 11/919,374

(22) PCT Filed: Apr. 24, 2005
(86) PCT No.: PCT/CN2006/000777

§ 371 Date: Oct. 26, 2007

(30) Foreign Application Priority Data
Apr. 27, 2005 (CN) 200510011636.7

(51) Int. Cl. A61B 5/145

(52) U.S. Cl. 600/365

(57) ABSTRACT

A diabetes monitor can provide diabetes information is invented in this invention. It comprises an input module, a storage module, and a knowledge base system module. This invention breaks through the design idea of traditional diabetes monitors. It not only can monitor the blood glucose values but also can generate the diabetes state of the patient. So it makes patients recognizing their own diabetes state in time, and can take effective control and treatment accordingly.

### THE TECHNICAL AREA

A device that can provide diabetes information is invented in this invention. And it is a diabetes monitor.

### TECHNICAL BACKGROUND

Currently, the prevention and treatment of diabetes is an important medical problem in the modern society. As the world Health Organization (WHO) reported, in 2003, there were 40 million people with diabetes in China, and the number of diabetes patients is still increasing at high speed. The severe damage to diabetes patients is the complications in the later stage, such as cerebrovascular lesion, retina lesion, renal disease, stroke and even amputation. At present time, there is no therapy to cure diabetes completely and radically. But through effective monitoring, food planning, physical activities, and medical treatment, the patient's condition can be controlled. WHO and all the doctors in the world emphasized that self-monitoring by patients themselves is the best way to control diabetes and to prevent diabetes complications.

At present time, people have taken the blood glucose monitor (BGM) as the monitoring tool of diabetes in China and in the whole world. Patients got knowledge of their diabetes situation from their blood glucose value (BGV) measured from the BGM. But the level and severity of diabetes of patients can not be determined by a single BGV in many cases. And it can always be determined by data of different BGVs.

Therefore, self-monitoring of BGVs is an effective method for patients to determine their own DS. And so they can control their diabetes situation effectively.

### Content of Invention

The goal of this invention is to provide a diabetes monitor that will make patients getting their own diabetes situation information through measuring their own BGVs. Moreover it provides the quantitative references for the control and treatment of their diabetes.

The technical plan of the diabetes monitor in this invention is as fellows:

A diabetes monitor comprises an input module, a storage module, and a knowledge-based system (KBSM) module. The storage module receives from the input module patients' BGVs, will be stored as arrays of BGVs according to different categories, and sent to the KBSM module, which will generate diabetes information by its inference capabilities based on the arrays of BGVs.

The input module is a BGV monitoring device that can monitor BGVs of patients.

The KBSM module is also connected with a display module, which can display the arrays of BGVs and/or the information of diabetes states.

The arrays of BGVs comprise BGVs of different categories, which are classified by sampling mode and/or sampling time. Different sampling modes comprise plasma venous glucose and capillary blood glucose; Different sampling time comprises fasting glucose, BGV-2hpm, BGV-2hpg, and casual glucose.

The KBSM module comprises a receiving unit, and an inference unit connected to the diabetes knowledge base (DKB), and an output unit. The DKB is a two-dimensional array describing the relationship between BGVs and DSs. The receiving unit receives the arrays of BGVs in the storage module. The inference unit compares the input data with all data in the two-dimensional array of the DKB and finds out the closest value with it in the DKB, which links the required information of diabetes.

Another technical plan of the diabetes monitor in this invention is as fellows:

A diabetes monitor comprises an input module, a storage module, and a knowledge-based system (KBSM) module. The storage module receives from the input module patients' BGVs, which will be stored as arrays of BGVs according to different categories, and sent to the data transmission module, which is connected to an outside KBSM module through data communication. And the KBSM module will generate diabetes information by its inference capabilities based on the arrays of BGVs.

The input module is a BGV monitoring device that can monitor BGVs of patients.

The arrays of BGVs comprise BGVs of different categories, which are classified by sampling mode and/or sampling time. Different sampling modes comprise plasma venous glucose and capillary blood glucose. Different sampling time comprises fasting glucose, BGV-2hpm, BGV-2hpg, and casual glucose, which is the BGV measured at the time excepting the above-mentioned three.

The KBSM module comprises a receiving unit, and an inference unit connected to the diabetes knowledge base (DKB), and an output unit. The DKB is a two-dimensional array describing the relationship between BGVs and DSs. The receiving unit receives the arrays of BGVs in the storage module. The inference unit compares the input data with all data in the two-dimensional array of the DKB, finds out the closest value with it in the DKB, and gets the corresponding diabetes information. The information will be sent to the display module to display and/or to the storage module to store through the data transmission module.

The above-mentioned data transmission module is a wireless communication terminal, an internet communication terminal, or a data interface connector.

Based on the stored BGV array and the DKB, this invention can generate the diabetes information and implement the monitoring of patient's diabetes states accurately. The technical plan of this invention breaks through the design idea of a traditional diabetes monitors. It not only monitors patients' BGVs, but also generates patients' diabetes states, which are required by the diabetes patients and the human society. The result the patient most interested in is not the measured BGV itself, but is the information of diabetes states generated from the measured BGVs. In fact, there is no simple linear relationship between the measured BGVs and the diabetes states, and hence the fully understanding of the diabetes states cannot be generated by a single test of a BGV. The diabetes state will be displayed on the screen of the diabetes monitor directly in this invention. And so the patients can get the knowledge of their own diabetes states, and can take necessary control or cure process, which not only can control the diabetes state effectively but also help patients' convalescence.

### EXPLANATION OF ATTACHED FIGURES

FIG. 1 is the schematic diagram of the structure of this invention;

FIG. 2 is the flow chart of the KBSM module of this invention;

FIG. 3 is the schematic diagram of another technical plan of the structure of this invention

### DETAILED DESCRIPTIONS OF ATTACHED FIGURES

The following is the detailed explanation of the technical plan by the attached figures:

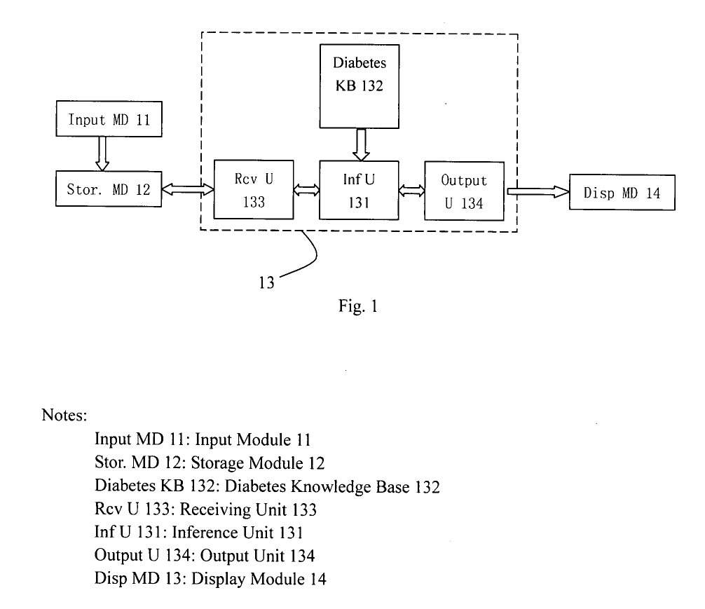

FIG. 1 is the schematic diagram of the structure of this invention. As shown in FIG. 1, the invention of the Diabetes Monitor comprises the input module 11, the storage module 12, the KBSM module 13 and the display module 14. Said KBSM module 13 comprises the receiving unit 133, the inference unit 131 connecting with the DKB 132, and the output unit 134.

The function of the input module 11 is taking patients' BGVs and sending them to the storing module 12. The input module 11 is a blood glucose monitor, a recording device, or a data interface connector. The input module 11 in the optimal plan is a blood glucose monitor, like the blood glucose monitor in the market, which measures the BGVs directly. After measuring the BGVs, the blood glucose monitor sends the data to the storage module 12. This data input can also be completed by a keyboard (an input device) or a data cable (a data interface connector) too.

Values stored in the storage module 12 are arrays of BGVs of different categories. In this example, the BGVs of patient is classified by sampling modes and sampling times; said sampling modes are samples taken from veins and capillary vessels. Different sampling times comprise fasting glucose, BGV-2hpm, BGV-2hpg, and casual glucose. Actually, the arrays of BGVs stored in the storage module 12 may comprise any combinations of the above-mentioned BGVs. The storing manner of the storage module 12 in this invention is different from that commonly used in the blood glucose monitor in the market. Obviously, patient's diabetes states can be generated only by taking BGVs. of different categories in many cases.

The KBSM module 13 receives the stored arrays of BGVs in the storage module 12 and generates the corresponding information of diabetes, which comprises hypoglycemia, normal state, Impaired Fasting Tolerance (IFG), Impaired Glucose Tolerance (IGT) and diabetes. The above 5 results describe the severity of diabetes and give patients references of taking necessary treatment.

Said KBSM module 13 comprises the receiving unit 133, the output unit 134, and the inference unit 131, which is connected with the DKB 132. The DKB 132 is a two-dimensional array describing the relationship between BGVs and diabetes states. The receiving unit 133 receives BGVs from the storage module 12. And the inference unit 131 searches the DKB 132 and finds out, in the DKB, the nearest value with the sample BGV, and so to find out the diabetes state information corresponding to these BGVs. The information will be displayed in the display module 14 and stored in the storage module 12.

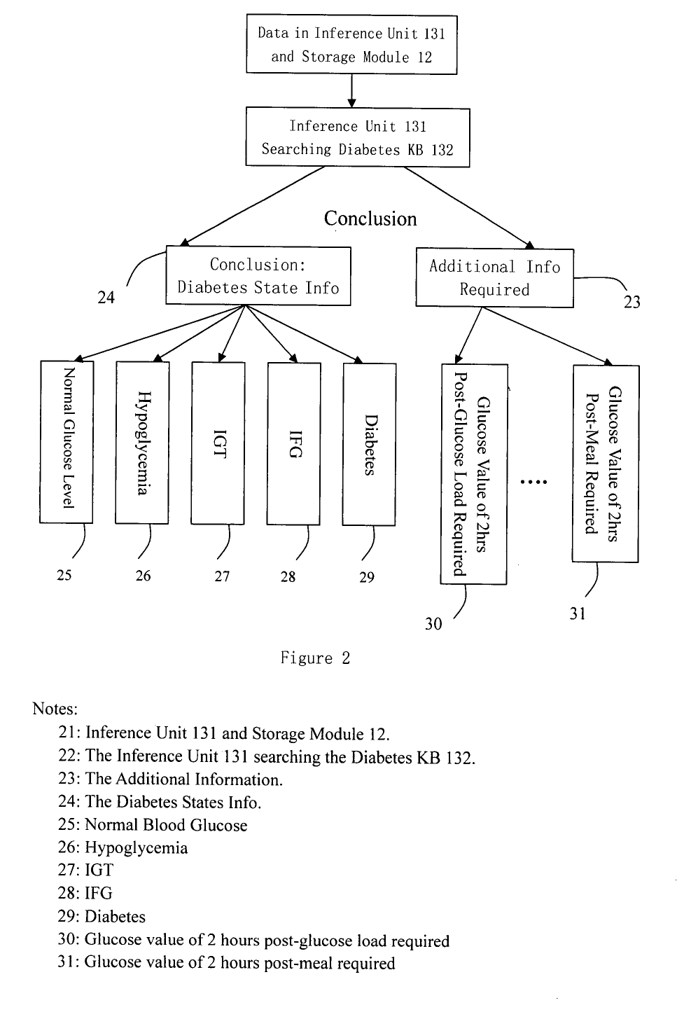

The DKB 132 stores a two-dimensional array which describes the relationship between the diabetes states and their corresponding BGVs. The two-dimensional array was built up according to the standard that was given by WHO and edited and published by China Diabetes Association. The standard is the bible to determine diabetes states from BGVs. The design of this invention takes the above document as reference, and classifies the sampling modes into taking blood from veins and from capillary vessels; classifies the sampling times into fasting, 2hpm, 2hpg, and casual. Each kind of BGVs is divided into 4-5 levels, so more than 400 BGVs combinations can be generated. According to the clinical experience, about 100 combinations are finally considered. Corresponding to these combinations, five different diabetes states, hypoglycemia, glucose normal, IFG, IGT, and diabetes, which corresponding blocks 27 through 31 in FIG. 2, are generated.

Sometimes, the numbers of the input glucose values are insufficient to determine a single diabetes state, additional information, such as “glucose value of 2hpm required”, “glucose value of 2hpg required”, etc. are displayed.

Different combinations of BGVs can be received by the inference unit 131, all combination cases have to be considered. Once the BGVs received by the inference unit 131 are insufficient to generate the information of illness, messages of “information insufficient” or “BGVs should be supplied” will be displayed. For example, if the fasting BGVs received by the inference unit 131 is 6.6 mmol/l, and 2 hours post-meal glucose is 8.5 mmol/l, the inference unit will give the tip of “2 hours post-glucose load required”.

The work flow in the KBSM module 13 in FIG. 2 can be completed through a software or a hardware chip, which might be a MCU. Depending on powerful abilities of the MCU, this invention can perform some other functions, such as:
(1) Create a diabetes treatment database, which can provide diabetes treatment information as references for patients.
(2) Create an alarm system, which can deliver a sound and/or a light alarm, once the critical point of severity of diabetes is reached.
(3) Create a communication interface connector, which can transmit diabetes states information and/or receive related documents.

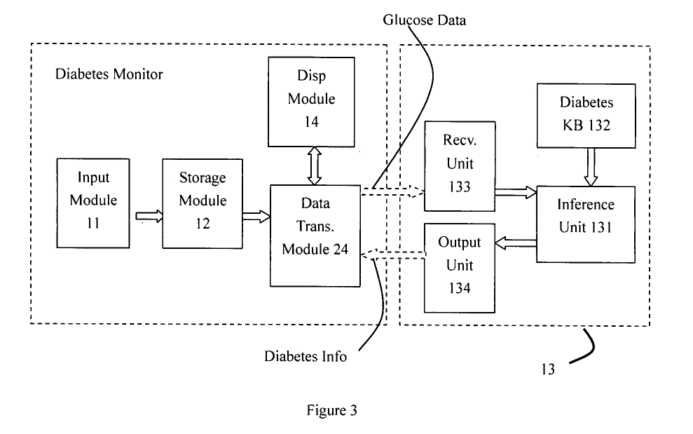

FIG. 3 is the schematic diagram of the structure of another technical plan in this invention. As shown in FIG. 3, this invention comprises the input module 11, the storage module 12, the data transmission module 24, and the display module 14. The input module 11 takes BGVs of patients and sending them to and storing in the storage module 12. The input module 11 may be the regular blood glucose monitor in the market. In this case, the BGVs of the patient can be classified by the sampling mode and sampling time. Sampling modes comprise blood from veins and from capillary vessels, and sampling times can be fasting, 2hpm, 2hpg, and casual. The array of BGVs is the combination of the BGVs mentioned above.

The key point of this technical plan is the creation of the data transmission module 24, which is connected with the storage module 12 and the display module 14. The data transmission module 24 is also connected with the KBSM module 13 through data communications. The above-mentioned data transmission module 24 transmits the arrays of BGVs from the storage module 12 to the KBSM module 13; and transmits diabetes information from the KBSM module 13 to the display module 14 and/or the storage module 12.

The structure and working mechanism of the outside KBSM module 13 are the same as we mentioned above, and the diabetes information comprises glucose normal, hypoglycemia, IFG, IGT, and diabetes.

The data transmission module 24 can be a wireless communication terminal or an internet communication terminal, or a data interface connector.

The wireless communication terminal is a data transmission device, which transfers data through a transportable communication network, such as a mobile phone. In this plan, the short message terminal communicates with the KBSM module 13 through the transportable communication network. The short message terminal sends the array of BGVs to the KBSM module 13 in the form of short message. After inference, the KBSM module 13 sends the diabetes information in the form of short message back to the short message terminal. The short message terminal transmits the diabetes information to the display module 14 to display and/or transmits to the storage module 12 to store.

The internet communication terminal is a device that transfers data through INTERNET network; it is a real time communication terminal, such as a laptop computer. In this plan, the real time communication terminal and the KBSM module 13 communicate with each other by the INTERNET network. The real time communication terminal sends the array of BGVs to the KBSM module 13 in the form of short message; and after inference, the KBSM module 13 sends the diabetes information in the form of short message back to the real time message terminal. The real time communication terminal transmits the diabetes information to the display module 14 to display and/or transmits to the storage module 12 to store. The real time communication is the output and input process between the computer network terminal users, such as the MSN of the Microsoft. Doubtless, these functions can also be performed by Email.

The data interface connector is the device that makes the data exchange with computers directly, such as I/O interface. In this plan, the computer that stores the KBSM module 13 receives the BGVs through the I/O interface. And after inference process, the KBSM module 13 sends back the diabetes information through the same I/O interface to the display module 14 to display and/or to the storage module 12 to store.

### Acronym

2hpg: 2 hours post-glucose load
2hpm: 2 hours post-meal

### BGM: Blood GlucoseMonitor

### BGV: Blood GlucoseValue

BGV-2hpg: BGV of 2 hours post-glucose load
BGV-2hpm: BGV of 2 hours post-meal

### DKB: Diabetes Knowledge Base

### DS: Diabetes Dtate

### IFG Impaired Fasting Tolerance

### IGT: Impaired Glucose Tolerance

### KB: Knowledge Base

### KBSM: Knowledge-based System

OGTT: Oral Glucose Tolerance Test

I claim:

1. A Diabetes Monitor comprising,
a set of input means receiving a set of patient's blood glucose values;
a set of storage means storing said set of patient's blood glucose values from said set of input means; and
a set of knowledge-based system means inferring information of diabetes states from said set of patient's blood glucose values received from said set of storage means.

2. Said set of input means of claim 1 is a set of blood glucose monitoring means for monitoring said set of patient's blood glucose values.

3. Said set of knowledge-based system means of claim 1 connects to a set of display means for displaying said set of patient's blood glucose values and said information of diabetes states.

4. The differences between said set of patient's blood glucose values of claim 1 is the differences of sampling times.

5. Said set of patient's blood glucose values of claim 4 comprising,
a set of fasting blood glucose values,
a set of blood glucose values by sampling times related to OGTT glucose load,
a set of blood glucose values by sampling times related to meal, and
a set of casual blood glucose values sampled at any time except the above mentioned three sets.

6. Said set of blood glucose values by sampling times related to OGTT glucose load of claim 5 being a set of blood glucose values sampled 2 hours post-glucose load; said set of blood glucose values that sampling time related to meal being a set of blood glucose values sampled 2 hours post-meal.

7. Said set of knowledge-based systems of claim 5 comprising,
a set of receiving means,
a set of output means, and
a set of inference means connected with a set of knowledge bases;
said set of knowledge bases being a set of two-dimensional arrays for describing relationship between said set of patient's blood glucose values and said set of diabetes states;
said set of receiving means receiving said set of patient's blood glucose values from said set of storage means;
said set of inference means searching said set of diabetes knowledge bases and comparing components in said set of knowledge bases with said set of received patient's blood glucose values and finding out one or more component that closest to received blood glucose values;
after getting diabetes states information, said set of knowledge-based system sending said information to said set of display means to display and/or said set of storage means to store.

8. Said Diabetes Monitor of claim 1 comprising
a set of input means, a set of storage means, a set of data transfer means, and a set of display means;
said set of storage means receiving said set of patients blood glucose values from said set of input means, storing them in the format of blood glucose value arrays, and delivering them to a set of data transfer means;
through data communications, said set of data transfer means connecting to a set of outside knowledge base means, delivering said set of patient's blood glucose value arrays to and receiving information about diabetes states from said set of knowledge-based systems.

9. Said set of input means of claim 8 is a set of blood glucose monitoring means for determining patient's blood glucose values.

10. Said Diabetes Monitor of claim 8 comprising said blood glucose array comprising blood glucose values of different categories; Said set of blood glucose values stored in said arrays of claim 8 comprising blood glucose values of different categories,
Said different categories comprising different sampling modes and different sampling times;
said different sampling modes comprising sampling blood from veins and from capillary vessels, and
said different sampling times comprising fasting blood glucose, sampling time relating to OGTT time, sampling time relating to meal, and any sampling times except said above-mentioned three times.

11. Said set of blood glucose values of claim 10 comprising
said set of blood glucose values related to OGTT being the blood glucose value-2hpg,
said blood glucose value related to the time of taking meal being the blood glucose value-2hpm, and
said casual blood glucose value being blood glucose value taking at any time except said blood glucose values-2hpg, said blood glucose value-2hpm, and said fasting blood glucose value.

12. Said set of outside knowledge-based system of claim 11, comprising
said set of receiving means,
said set of inference means that connected with said setoff diabetes knowledge base,
said set of output means;
said set of diabetes knowledge bases being a set of two-dimensional arrays describing relationships between said set of blood glucose value array and said set of diabetes states;
said set of receiving units receiving said set of blood glucose values from said set of storage means;
said set of inference means searching said set of diabetes knowledge bases and comparing components in said knowledge base with said received blood glucose values and finding out one or more component that closest to said received blood glucose values; wherein getting diabetes states information, sending to said display means to display and/or to said storage means to store.

13. Said set of data transmitting means in said diabetes monitor of claims 12, being a set of wireless communication terminals, internet communication terminals, and data interfaces.
